(19) United States
(12) Patent Application Publication
(10) Pub. No.: US 2017/0336404 A1
(43) Pub. Date: Nov. 23, 2017

(54) RAPID IMMUNOASSAYS

(71) Applicant: Alpha Diagnostic International, Inc., San Antonio, TX (US)

(72) Inventor: Masarrat Ali, San Antonio, TX (US)

(21) Appl. No.: 15/599,602

(22) Filed: May 19, 2017

Related U.S. Application Data
(60) Provisional application No. 62/339,037, filed on May 19, 2016.

(51) Int. Cl. G01N 33/558; G01N 33/543

(52) U.S. Cl. CPC G01N 33/558; G01N 33/54386

(57) ABSTRACT

Provided herein are methods and devices capable of depleting cross-reactive species from a test sample as it travels through the absorbant substrate of a rapid immunoassay test. The methods and devices disclosed herein enable the reduction or elimination of cross-reacting materials without the need for any additional user steps in using the rapid test.

### CROSS-REFERENCE TO RELATED APPLICATIONS

This application claims the benefit of U.S. Provisional Application No. 62/339,037, filed May 19, 2016, the disclosure of which is hereby incorporated by reference in its entirety.

### BACKGROUND

The immunoassay is one of the most common analytical techniques for identification of specific proteins, peptides, hormones, antibodies, and many other types of substances from a complex sample. A popular immunoassay format is the so-called “rapid test.” A home-use pregnancy testing kit is one example of a rapid immunoassay test.

Typically, rapid tests employ the immunoassay technique to provide a fast result after applying a sample in the sample application area of the device. The device itself is typically a cassette or a cartridge housing an absorbent substrate strip. The housing leaves the strip exposed in two locations: a sample application well at the proximal end and a results window in the center to distal end of the cartridge. After being applied to the sample well, the sample wicks through the solid-phase substrate, where it interacts with specific immobilized reagents applied as stripes on the substrate. The interaction of the sample with the reagents generates a detectable result, such as a visible color change, indicating a positive or negative test result. In most common tests, the results are typically visible as one or more stripes in the results window of the device, with one stripe typically indicating that the device is working as expected, even in the case of a negative test result, and another stripe indicating the test result.

In some cases, rapid immunoassay tests may suffer from issues related to cross-reacting materials that can lead to confusing or erroneous results. For example, Zika virus is very closely related to a host of other viruses such as West Nile virus, Dengue virus, Japanese Encephalitis virus, and others, all belonging to the same family of viruses. Another related virus is Chikungunya virus, which causes disease symptoms similar to Zika virus but belongs to a different family of viruses. Due to the relatively high degree of conservation in the sequences of specific antigenic proteins from these viruses, it is very difficult to raise highly specific reagents for use in immunoassays. In the case of testing for exposure to Zika virus, false positives may arise from the presence of cross-reactive substances in the sample. For example, current testing methods cannot distinguish specific antibodies against Zika virus within the sample from cross-reacting antibodies (from a previous exposure) to Dengue virus or West Nile virus.

There are other cases where the objective of a test is to identify range of different mutations within proteins that are associated with disease phenotypes against the background of normal protein in the sample in the absence of highly specific immunoassay reagents. Another example is where the objective of the test is to identify modified protein against the background of unmodified protein using reagents that exhibit cross-reactivity. In all of the above examples, the use of immunoassay techniques with the rapid test format often provides ambiguous results. There thus exists a need for methods and devices capable of decreasing ambiguities caused by cross-reactive materials in immunoassays.

### SUMMARY

In one aspect, the disclosure provides rapid immunoassay test devices comprising a test strip, the test strip comprising: (a) a proximal end comprising a sample receiving zone; (b) a cross-reactive species binding zone positioned downstream of the sample receiving zone, the cross-reactive species binding zone comprising at least one cross-reactive species binding reagent that is immobilized on the test strip; and (c) a target species binding zone positioned downstream of the cross-reactive species depletion zone, the target species binding zone comprising at least one target binding reagent and one or more target detection reagents enabling detection of a target species bound to the target binding reagent.

In some embodiments, the cross-reactive species binding zone comprises a quantity of cross-reactive species binding reagent sufficient to substantially deplete a sample of cross-reactive species. In some embodiments, the rapid immunoassay test devices of the disclosure further comprise one or more cross-reactive species detection reagents enabling detection of cross-reactive species bound by the cross-reactive species binding reagent.

In some embodiments, the interaction of the one or more target detection reagents with the target species and/or the target species binding reagent produces a visible signal. In some embodiments, the interaction of the one or more cross-reactive species detection reagents with the cross-reactive species and/or the cross-reactive species binding reagent produces a visible signal.

In some embodiments, the test strip is disposed within a housing comprising a sample application area in fluid communication with the sample receiving zone of the test strip. In some embodiments, the housing further comprises a viewing window through which the target species binding zone is visible. In some embodiments, the housing further comprises a viewing window through which the cross-reactive species binding zone is visible.

In some embodiments, the test strip is made of absorbent material and is adhered to a backing member.

These as well as other aspects, advantages, and alternatives, will become apparent to those of ordinary skill in the art by reading the following detailed description, with reference where appropriate to the accompanying drawings, and taken together with the accompanying claims.

### BRIEF DESCRIPTION OF THE DRAWINGS

The following detailed description of the embodiments of the present invention can be best understood when read in conjunction with the following drawings, in which:

FIG. 1 shows an exemplary rapid test device cartridge of the disclosure with a sample application well and test window, and the substrate within the cartridge with various capture agents applied as stripes.

FIG. 2 shows a schematic representation of the different capture agents applied onto a rapid test device substrate of the disclosure and the corresponding substances within a sample that would bind to the capture agents on the substrate.

FIG. 3 shows a close-up view of an exemplary rapid test device containing the different capture agents striped at specific locations on the substrate.

FIG. 4 shows an exemplary result of applying to a rapid test device of the disclosure a sample containing cross-reacting substances in addition to a substance of interest.

FIG. 5 shows an exemplary result of applying to a rapid test device of the disclosure a sample containing cross-reacting substances but devoid of a substance of interest.

### DETAILED DESCRIPTION

Provided herein are methods and devices capable of depleting cross-reactive species from a test sample as it travels through the absorbant substrate of a rapid immunoassay test, so that the result provided is less likely to be a false positive or a false negative. The methods and devices disclosed herein enable the reduction or elimination of cross-reacting materials without the need for any additional steps in using the rapid test; thus, the methods and devices of the disclosure do not interfere with a rapid immunoassay's ability to provide a fast, visually discernible result.

All publications, patents and patent applications cited herein are hereby expressly incorporated by reference for all purposes.

Before describing the disclosed methods and devices in detail, a number of terms will be defined. As used herein, the singular forms “a”, “an”, and “the” include plural referents unless the context clearly dictates otherwise. For example, reference to a “sample” means one or more samples.

It is noted that terms like “preferably,” “commonly,” and “typically” are not utilized herein to limit the scope of the claimed aspects and embodiments or to imply that certain features are critical, essential, or even important to the structure or function of the claimed aspects and embodiments. Rather, these terms are merely intended to highlight alternative or additional features that can or cannot be utilized in a particular embodiment.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Exemplary embodiments are described herein. It should be understood that the word “exemplary” is used herein to mean “serving as an example, instance, or illustration.” Any embodiment described herein as “exemplary” is not necessarily to be construed as preferred or advantageous over other embodiments. Further, those skilled in the art will understand that changes and modifications may be made to these embodiments without departing from the true scope and spirit of the invention, which is defined by the claims.

As used herein, the terms “rapid immunoassay” or “rapid immunoassay test” refer to a biochemical assay that measures the presence or concentration of molecule (such as a macromolecule or small molecule) a solution or sample through the use of an antibody or an antigen, and which furthermore provides a result in a rapid fashion, such as in less than 20, or less than 15 minutes, or less than 10 minutes, or less than 9 minutes, or less than 8 minutes, or less than 7 minutes, or less than 6 minutes, or less than 5 minutes, or less than 4 minutes, or less than 3 minutes, or less than 2 minutes, or less than 60 seconds, or less than 30 seconds. In some embodiments, the rapid tests of the disclosure provide a result in from about 30 seconds to about 10 minutes. In some embodiments, the rapid tests of the disclosure provide a result in less than about 5-10 minutes. In some embodiments, the rapid tests of the disclosure provide a result in 30 seconds to 2 minutes. In some embodiments a substrate with a slower fluid mobility rate may be advantageous to provide for more time for cross-reactive substances to interact with the cross-reactive species binding agents.

Any rapid immunoassay format is contemplated to be compatible with the methods disclosed herein. A particular rapid immunoassay format contemplated to be compatible with the methods and devices disclosed herein is the so-called “lateral flow test” or “lateral flow immunochromatographic assay,” which is a test (typically embodied in a device) capable of detecting the presence (or absence) of a target analyte in sample (matrix) without the need for specialized and costly equipment, though many lab based applications exist that are supported by reading equipment. Typically, lateral flow tests are used for medical diagnostics either for home testing, point of care testing, or laboratory use. A home pregnancy test is an example of a lateral flow test.

Typically, a lateral flow test device comprises one or a plurality of capillary beds composed of, for example, porous paper, microstructured polymer, or sintered polymer. Each capillary bed has the capacity to absorb and/or transport fluid (e.g., urine) spontaneously, such as through capillary action (e.g., wicking). Typically, a sample is delivered to a sample pad or sample delivery region, which acts as a sponge and holds an excess of sample fluid. Once soaked, the fluid migrates to a target binding region (sometimes contained on a separate but adjoining pad in fluid communication with the sample delivery region) that contains a dried format of bio-active reagents in a matrix, such as a salt-sugar matrix, which comprises everything to guarantee an optimized chemical reaction between the target molecule (e.g., an antigen) and its chemical partner (e.g., antibody). In some cases the chemical binding partner (i.e. target binding reagent) is immobilized within the capillary pad itself. In other cases, the binding partner or target binding reagent is conjugated to or immobilized on the surface of a particle embedded or dried onto the capillary bed. When the sample fluid reaches the target binding region, the fluid dissolves the matrix and in one combined transport action the sample and bioactive reagents mix while flowing through the porous structure. In this way, the analyte binds to the target binding reagent while migrating.

Typically, a lateral flow test further comprises detection reagents within or downstream of the target binding region. The detection reagents are typically disposed in one or more areas (often called stripes) in which a detection molecule has been immobilized upon or applied to the test strip by the manufacturer. Once the sample-conjugate complex reaches the detection reagents, the detection reagent binds the complex. The detection reagent-sample-conjugate complex forms a visible moiety, such that as sample fluid passes over or through the detection stripes, detection reagent-complex particles accumulate in a defined area and the stripe-area changes color. Typically there are at least two stripes: one (the control) that captures any particle and thereby shows that reaction conditions and technology worked as expected, and the second (or more) detects only sample-conjugate complexes. Typically, after passing these reaction zones the fluid enters the final porous material, sometimes called the wick, which simply acts as a waste container.

In cases where the target binding reagent is immobilized on a particle, in principle any colored particle can be used. Commonly used particles are made from latex (blue color) or nanometer sized particles of gold (red color). Gold nanoparticles are red in color due to localised surface plasmon resonance. Fluorescent or magnetic labeled particles can also be used, which typically require the use of an electronic reader to assess the test result. In some cases, the material used for the particle will determine the operation of the control stripe. For example, where latex or gold particles are used, the control stripe may, for example, comprise an antibody or other capture reagent that binds to and captures free latex/gold in order to confirm the test has operated correctly.

In other embodiments, detection of a target analyte within a lateral flow test can be based on chemiluminescence, such as the reaction between horseradish peroxidase and luminol. For example, rather than being conjugated to a colored particle, the target species binding agent may be conjugated to horseradish peroxidase, which reacts with luminol that has been added to the sample fluid or is otherwise present on the test strip to form light. Thus, when the target species has bound to the target binding reagent (which itself is conjugated to horseradish peroxidase), and then passes over or through the detection stripe and is bound to a detection reagent immobilized within the detection stripe, horseradish peroxidase conjugates are thereby concentrated within the detection stripe and yield a detectable chemiluminescent signal.

Lateral flow tests can operate as, for example, competitive assays or sandwich assays. In a sandwich assay, the sample encounters coloured particles which are labeled with antibodies raised to the target analyte. The detection stripe comprising detection reagents will also contain antibodies to the same target, although it may bind to a different epitope on the analyte. As a result, the detection stripe will show as a colored band in positive samples. An example of the sandwich assay is the sandwich enzyme-linked immunosorbent assay (ELISA).

An example of a competitive lateral flow test, the target analyte itself or an analog is immobilized on colored particles. The detection stripe contains antibodies to the target or its analog. Free target analyte in the sample then acts to block the binding sites on the antibodies preventing binding of the colored particles to the detection stripe. Thus, a detection stripe in a competitive lateral flow test will show as a coloured band in negative samples.

Although lateral flow tests typically yield qualitative results, in some cases the intensity of the detection stripe can be quantitatively assessed to determine the quantity of analyte in the sample. Handheld diagnostic devices known as lateral flow readers may be used to provide a fully quantitative assay result. By utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. Alternative non-optical techniques are also able to report quantitative assays results. One such example is a magnetic immunoassay (MIA) in the lateral flow test form also allows for obtaining quantitative results.

As used herein, the terms “zone” and “region” are interchangeable and refer to an area of an object or device, such as a test strip, which is typically identified by the purpose served by that area. Thus, a sample receiving zone or sample receiving region denotes an area of a device disposed to receive a sample.

As used herein, the terms “reagent” and “agent” are interchangeable and refer to any substance, chemical entity, or biochemical entity for use in chemical, biochemical, or biological analysis. Thus, the terms “binding reagent,” “binding agent,” “capture reagent,” and “capture agent” are interchangeable and refer to chemical or biochemical entites capable of binding to a given species within a sample for analytical purposes. Antibodies are examples of binding/capture reagents.

As used herein, the term “target species” refers to one or more species within a test sample, the detection of which is the primary purpose of a test, experiment, or assay. For example, a home pregnancy test might be designed to test for the presence of human chorionic gonadotropin (hCG) in a blood or urine sample, in which case the target species is hCG. Rapid immunoassays can be adapted to detect a wide variety of target species, including proteins, nucleic acids, and small molecules.

As used herein, the term “target species binding reagent” refers to a reagent capable of binding to a target species. For example, if a target species is hCG, a target species binding reagent would be any reagent capable of binding to hCG, such as an anti-hCG antibody. As another non-limiting example, if the target species within a sample is an antibody, such as an antibody against a particular viral antigen, such as an anti-NS1 (non-structural protein 1) antibody, a target species binding reagent would be a reagent capable of binding to an anti-NS1 antibody, such as the NS1 antigen itself. If the target species is a nucleic acid, the target species binding reagent could, for example, be a complementary nucleic acid capable of hybridizing with the target species. In some embodiments, the target species binding reagent is immobilized on a particle or bead, such as a microparticle or nanobead. In some embodiments, the particle or bead has a visible color. In some embodiments, the particle or bead is fluorescent. In some embodiments, the particle or bead is magnetic.

As used herein, the term “cross-reactive species” refers to any species within a test sample, other than a target species, that may bind to a target species binding reagent. For example, if a target species is hCG, a cross-reactive species would be any species present in the sample other than hCG that is also capable of binding to an anti-hCG antibody or other hCG binding reagent.

As used herein, the term “cross-reactive species binding reagent” refers to a reagent capable of binding to a cross-reactive species. For example, if a cross-reactive species is a protein that is related to, but distinct from a target protein, a cross-reactive species binding reagent would be any reagent capable of binding to the cross-reactive protein, such as an antibody specific for the cross-reactive protein, but which does not bind to the target protein. In some embodiments, a cross-reactive species binding reagent is immobilized on a particle or bead, such as a microparticle or nanobead. In some embodiments, the particle or bead has a visible color. In some embodiments, the particle or bead is fluorescent. In some embodiments, the particle or bead is magnetic.

In one aspect, the disclosure provides rapid immunoassay test devices comprising a test strip, the test strip comprising: (a) a proximal end comprising a sample receiving zone; (b) a cross-reactive species binding zone positioned downstream of the sample receiving zone, the cross-reactive species binding zone comprising at least one cross-reactive species binding reagent that is immobilized on the test strip; and (c) a target species binding zone positioned downstream of the cross-reactive species depletion zone, the target species binding zone comprising at least one target binding reagent and one or more target detection reagents enabling detection of target species bound to the target binding reagent.

In some embodiments, the cross-reactive species binding zone of the test strip comprises a quantity of cross-reactive species binding reagent sufficient to substantially deplete a sample of cross-reactive species. For example, in some embodiments, the cross-reactive species binding region comprises one or more binding proteins that are capable of capturing cross-reactive species from within a sample, thereby depleting the sample of cross-reactive species before the remainder of the species within the sample reaches the target species binding region of the test strip, where the sample interacts with a binding agent specific for the target species within the sample. In this manner, the cross-reactive species binding region lowers the chances that a cross-reactive species—rather than the target species—will interact with the target binding agent, which would result in a false positive (or false negative).

In some embodiments, the target species detection reagent binds specifically to a complex comprising the target species and the target species binding reagent. In some embodiments, the target species detection reagent is immobilized on the test strip of the disclosure in the form of a detection stripe. In some embodiments, interaction of the target species detection reagent with a complex of a target species and a target species binding reagent generates a visible signal. In some embodiments, interaction of the target species detection reagent with a complex of a target species and a target species binding reagent generates a signal that not necessarily visible to the naked eye, but is detectable using analytical instrumentation.

As a non-limiting example of a test strip of the disclosure, where the target species in a sample are antibodies against NS or envelope protein of the Zika virus, the cross-reactive species binding region has multiple stripes of antigens (e.g., NS1 or Env) from family members or other closely related viruses such as Dengue virus, West Nile virus, and Chickungunya virus. When a sample is applied to the test device, the sample migrates along the test strip by capillary wicking action and the NS antibodies within the sample interact with and are captured by (i.e., bind to) the NS antigens specific to the other viruses that are serially striped across the strip. The cross-reactive antibodies, if present, will be bound to the NS protein antigen stripes within the cross-reactive species binding region, leaving behind only those NS antibodies that are highly specific to the Zika virus NS protein to interact with and be captured by the Zika NS antigen stripe in the target binding region of the strip.

Thus, in some embodiments, the cross-reactive species binding zone comprises stripes of one or more cross-reactive species binding reagents which have been serially applied to the test strip. In some embodiments, the cross-reactive species binding zone comprises 1, or 2, or 3, or 4, or 5, or 6, or 7, or 8, or 9, or 10, or 11, or 12, or 13, or 14, or 15 different cross-reactive species binding reagents. In some embodiments, the cross-reactive species binding zone comprises from 1 to 5 different cross-reactive species binding reagents. In some embodiments, the cross-reactive species binding zone comprises a bolus mixture of one or more cross-reactive species binding reagents, which is applied to the test strip. In some embodiments, the cross-reactive species binding zone comprises both stripes of one or more cross-reactive species binding reagents and a bolus mixture of one or more cross-reactive species binding reagents applied to the strip. In some embodiments, the bolus mixture is positioned on the strip between the stripes of cross-reactive species binding reagents and the target binding region of the strip. In some embodiments, the bolus mixture is present on the strip to further deplete a sample of cross-reactive species before the sample reaches the target species binding region of the strip. In some embodiments, the bolus mixture is present on the strip between the sample application well and the cross-reactive species binding reagents.

Continuing the above example, in addition to the NS antigen stripes that are serially applied on the substrate, a bolus mixture of the NS antigens from the non-Zika viruses may be applied in the region between the cross-reactive species binding region and the target species binding region. This serves as an additional gatepost to further ensure that most or all of the cross-reacting antibodies are captured, leaving behind only the Zika-specific NS antibody to interact with the Zika NS antigen on the last stripe on the substrate within the target binding region.

In another example, the target species may be a protein with a specific mutation against a background of other variants of the same protein within a sample. In this case, the cross-reactive species binding reagents are, for example, capture antibodies for each of the variant proteins, which are striped on the substrate. The target binding region of the test strip comprises a capture antibody specific for the mutation of interest in the protein (i.e., the target species). Any cross-reactivity between the various striped antibodies in the cross-reactive species binding region and the protein variants within the sample are captured by the “gatepost” antibodies (i.e., the cross-reactive species binding reagents) leaving behind only that protein variant with the specific mutation to be captured and detected by the target species binding reagent contained in the target binding region of the test strip.

In another example, the target species may be a specific post-translational modification within a protein in a sample against a background of other modifications and unmodified protein. In such a case, the cross reactive species binding reagents are, for example, capture antibodies for unmodified and modified proteins other than the target species, which capture antibodies are striped on the substrate within the cross-reactive species binding region. The target species binding region contains one or more capture antibodies specific for capture of the modification of interest in the protein (i.e., target species binding reagents). In this case, any residual cross-reactivity between the various striped antibodies and the “gatepost” antibodies is depleted by the cross-reactive species binding reagents in the cross-reactive species binding region, leaving behind only the proteins containing the specific modification to be captured and detected by the target species binding reagent contained in the target binding region of the test strip.

In some embodiments, the cross-reactive species binding zone further comprises one or more cross-reactive species detection reagents enabling detection of cross-reactive species bound by the cross-reactive species binding reagent. In some embodiments, the cross-reactive species detection reagent binds specifically to a complex comprising the cross-reactive species and the cross-reactive species binding reagent. In some embodiments, the cross-reactive species detection reagent is immobilized on a test strip of the disclosure in the form of a detection stripe. In some embodiments, interaction of the cross-reactive species detection reagent with a complex of a cross-reactive species and a cross-reactive species binding reagent generates a visible signal. In some embodiments, interaction of the cross-reactive species detection reagent with a complex of a cross-reactive species and a cross-reactive species binding reagent generates a signal that not visible to the naked eye, but is detectable using analytical instrumentation.

In some embodiments the test strips of the disclosure are disposed within a housing comprising a sample application area in fluid communication with the sample receiving zone of the test strip. In some embodiments, the housing further comprises a viewing window through which the target species binding zone is visible. In some embodiments, the housing comprises a viewing window through which one or more stripes comprising target species detection reagents are visible. In some embodiments, the housing further comprises a viewing window through which the cross-reactive species binding zone is visible. In some embodiments, the housing comprises a viewing window or viewing windows through which one or more stripes comprising cross-reactive species detection reagents are visible.

The housing can be made from any material capable of being formed, machined, cast, etc. into the desired shape. In some embodiments, the housing is made from plastic.

In some embodiments, the test strip is made of absorbent material. In general, the test strip may be made from any material capable of absorbing a fluid sample, and along which a fluid sample may wick through or move through by capillary action. Non-limiting examples of materials from which test strips of the disclosure may be made from include porous paper, nitrocellulose, microstructured polymer, sintered polymer, porous ceramic, etc.

In some embodiments, the absorbent material of the test strip is adhered to a backing member. Typically, the backing member is itself non-absorbent and non-porous.

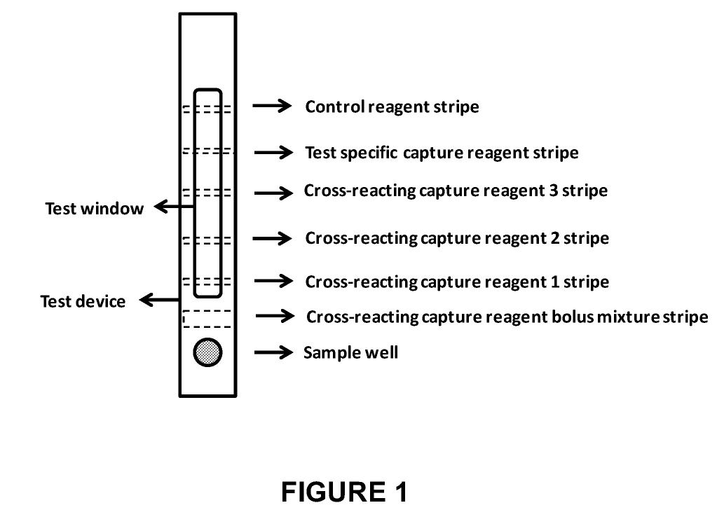

FIG. 1 illustrates an exemplary rapid immunoassay test of the disclosure comprising a cartridge containing a sample application well, a test window to visualize the results, and a substrate strip within the cartridge on which the various binding and detection reagents are embedded. In the figure, the capture/binding agents are applied as stripes on the substrate, which stripes also comprise a detection reagent that produces a visible color only when a specific interaction has occurred between the capture agent, the specific substances contained within the sample, and the detection reagent. Of the various capture agents that are striped on the substrate, the one that is most distal to the sample application well is a control reagent (capture agent 1) that produces a visual signal when a sample is applied. This control reagent serves to indicate that the test system is functioning as expected when a sample is applied. The capture agent stripe below the control reagent (capture agent 2) is specific for the substance of interest in the sample and will produce a visible signal when an interaction occurs with the specific substance within the sample. The remaining capture agent stripes, which could range from 1-3 different stripes (capture agents 3-5), are specific for the cross-reacting substances within the sample. In addition to the capture stripes on the substrate in the window region of the cartridge, the region between the sample application and well and the test window could also contain a bolus mixture of capture agents 3-5 in larger amounts that what is present on the individual stripes. The bolus capture agent mixture does not contain the detection reagent as it will not be visible through the cartridge.

When a liquid sample comprising the substance of interest along with several other cross-reacting substances is applied to the sample application well, the liquid migrates through the substrate material via capillary action and encounters the capture agents along its path. The cross-reacting substances will interact and bind to their specific capture agents as the liquid continues to migrate towards the distal end of the cartridge. Any cross-reacting substances that interact and bind to their corresponding capture agents will produce a visible color signal due to the presence of the detection reagent, thereby indicating the presence of the specific cross-reacting substances. When the sample eventually encounters the capture agent specific for the substance of interest, the sample will be devoid of any cross-reacting substances. The substance of interest will interact and bind to capture agent 4 and produce a visible signal due to the presence of the detection reagent. Lastly, the sample will continue migrating and eventually interact with the control capture agent which will produce a visible color that indicates that a sample was indeed applied to the test device. In other words, the control capture agent is a positive control for the proper functioning of the test device when a sample is applied. It should be noted that the device could also contain an optional bolus mixture of the capture agents specific for the cross-reacting materials (capture agents 3-5 in this example). This bolus mixture serves to additionally ensure that all cross-reacting materials are trapped from the sample, leaving behind only the substance of interest to interact with its specific capture agent and produce a signal. In this manner, the bolus mixture on the substrate and the capture agents 3-5 serve to deplete the sample of any cross-reacting substances, leaving behind only the substance of interest, if present in the sample, to be detected using the test device.

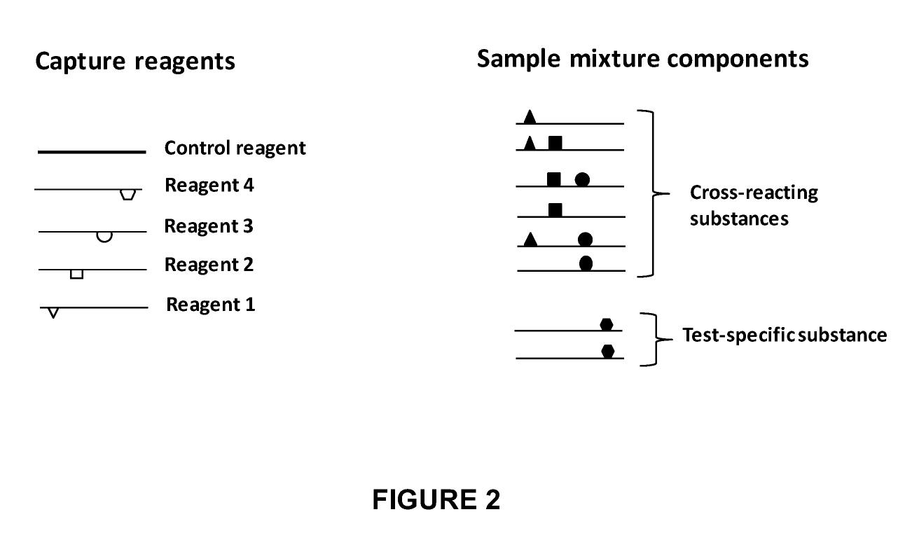

FIG. 2 schematically represents the the various capture agents, the control agent that are applied as stripes on the test device, and the various substances in the sample that comprise the cross-reacting substances as well the target species of interest. The individual capture agents that are specific for binding to a specific cross-reacting substance are indicated by the various geometrical shapes with the partial unfilled hexagon shape representing the capture agent that is specific for binding to the substance of interest. The control agent is represented a solid line which represents the common region that is present in all substances in the sample that are being tested in a particular test using this test device. Similarly, the various substances present in the sample that are being investigated in this test are schematically represented by the various filled geometric shapes. In this example, the substance of interest is indicated by the filled hexagon shape whereas the the cross-reacting substances are indicated by filled other shapes.

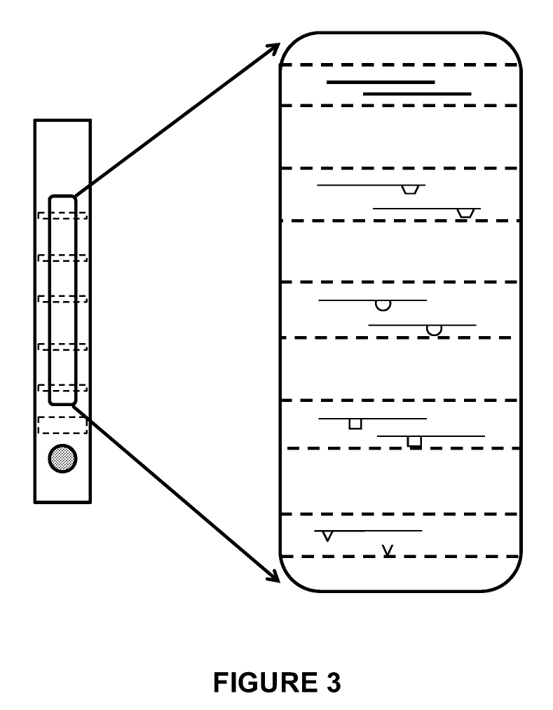

FIG. 3 illustrates the close-up design of the test device window showing the various capture agent stripes indicated by parallel dashed lined regions and the specific capture agents contained within the parallel dashed lines indicated by the the various geometric shapes as described in FIG. 2. In some embodiments, as depicted here, the capture agent stripes are applied such that they traverse the entire width of the substrate. This is done so that when the sample migrates longitudinally across the substrate, the capture agents have maximum opportunities to interact with the substances within the sample.

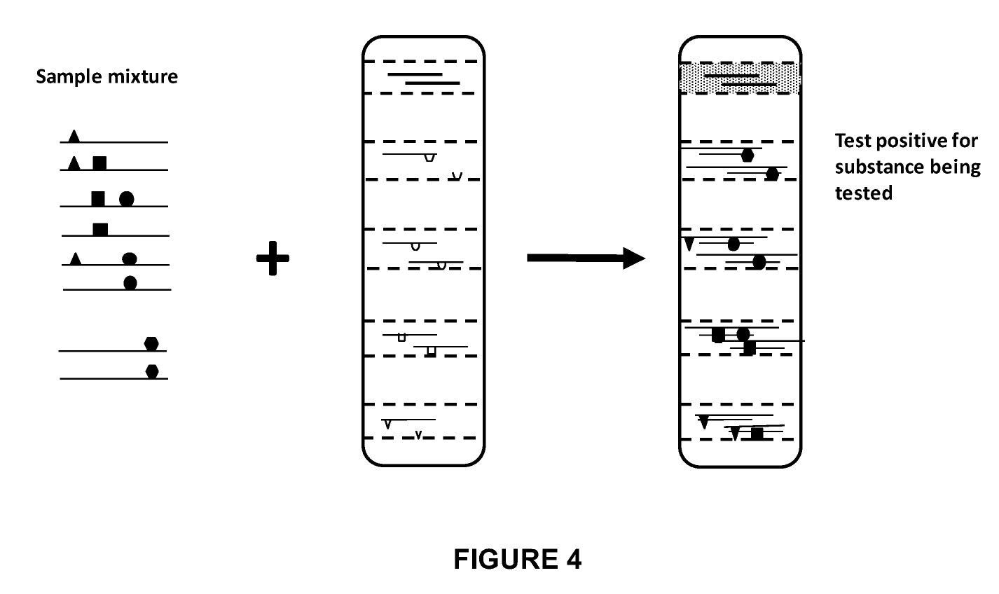

FIG. 4 illustrates an example of the use of the test device to deplete the cross reacting substances from a sample so that the substance of interest can be specifically detected. The test sample in this example contains multiple cross reacting species related to the target substance of interest as indicated by the various filled geometric shapes. The target species of interest that is present in this test sample is indicated by filled hexagons. When the sample is applied to the test device, it will migrate along the length of the substrate and the various cross-reacting substances will interact with the various capture agents and become immobilized. As the sample continues to migrate, it will be depleted of all the cross-reacting substances leaving behind only the substance of interest to interact and bind to its specific capture agent. In this example, the signal will be generated from all the capture agent locations including the control agent location, which is to be expected.

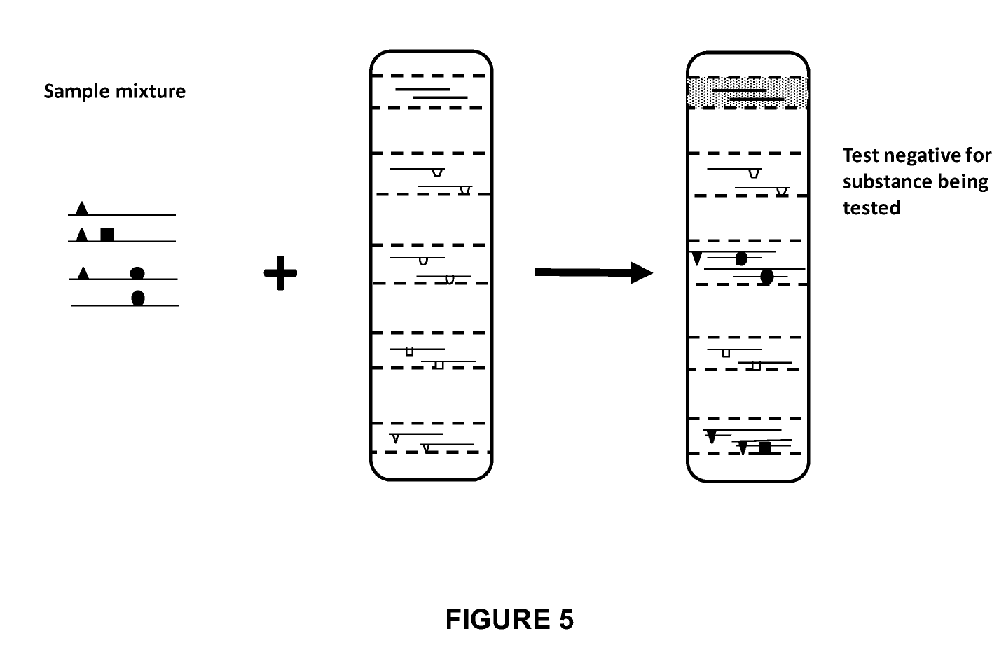

FIG. 5 illustrates a different example where certain cross-reacting substances are present within the sample that is known to be devoid of the substance of interest. In this example, when the sample is applied to the test device, it will migrate and function as described above, with the exception that the signal will only be generated from 2 of the 3 capture agents for the cross-reacting species with no signal from the capture agent that the specific for the substance of interest. This is because the sample contains only certain cross-reacting species of the substance and will therefore only interact and become immobilized at select capture agent locations on the substrate. The control agent will give a signal as expected, indicating that the device is functioning normally.

It should be understood the arrangements and functions described herein are presented for purposes of example only, and that numerous variations are possible. For instance, elements can be added, omitted, combined, distributed, reordered, or otherwise modified.

1. A rapid immunoassay test device comprising a test strip, the test strip comprising:
(a) a proximal end comprising a sample receiving zone;
(b) a cross-reactive species binding zone positioned downstream of the sample receiving zone, the cross-reactive species binding zone comprising at least one cross-reactive species binding reagent that is immobilized on the test strip; and
(c) a target species binding zone positioned downstream of the cross-reactive species depletion zone, the target species binding zone comprising at least one target binding reagent and one or more target detection reagents enabling detection of a target species bound to the target binding reagent.

2. The rapid immunoassay test device of claim 1, wherein the cross-reactive species binding zone comprises a quantity of cross-reactive species binding reagent sufficient to substantially deplete a sample of cross-reactive species.

3. The rapid immunoassay test device of claim 1, further comprising one or more cross-reactive species detection reagents enabling detection of cross-reactive species bound by the cross-reactive species binding reagent.

4. The rapid immunoassay test device of claim 1, wherein the interaction of the one or more target detection reagents with the target species and/or the target species binding reagent produces a visible signal.

5. The rapid immunoassay test device of claim 1, wherein the interaction of the one or more cross-reactive species detection reagents with the cross-reactive species and/or the cross-reactive species binding reagent produces a visible signal.

6. The rapid immunoassay test device of claim 1, wherein the test strip is disposed within a housing comprising a sample application area in fluid communication with the sample receiving zone of the test strip.

7. The rapid immunoassay test device of claim 6, wherein the housing further comprises a viewing window through which the target species binding zone is visible.

8. The rapid immunoassay test device of claim 6, wherein the housing further comprises a viewing window through which the cross-reactive species binding zone is visible.

9. The rapid immunoassay test device of claim 1, wherein the test strip is made of absorbent material and is adhered to a backing member.
